(19) United States
(12) Patent Application Publication
(10) Pub. No.: US 2024/0358924 A1
(43) Pub. Date: Oct. 31, 2024

(54) DEVICES, SYSTEMS, AND METHODS FOR COMBINING AND/OR DELIVERING INJECTABLE MATERIALS

(71) Applicants: Boston Scientific Medical Device Limited, Galway (IE); Boston Scientific Scimed, Inc., Maple Grove, MN (US)

(72) Inventors: Nitesh Ghananil Baviskar, Kalyan West (IN); Benjamin Cleveland, Bellingham, MA (US); Sanjay Kumar Chandra, Pune (IN); Katie Knowles, Providence, RI (US); Junaid Mohammed Shaikh, Surat (IN); Marshall O'Hearn, Davenport, FL (US)

(73) Assignees: Boston Scientific Medical Device Limited, Galway (IE); Boston Scientific Scimed, Inc., Maple Grove, MN (US)

(21) Appl. No.: 18/645,117

(22) Filed: Apr. 24, 2024

Related U.S. Application Data
(60) Provisional application No. 63/462,139, filed on Apr. 26, 2023.

(51) Int. Cl. A61M 5/24; A61M 5/19; A61M 5/315

(52) U.S. Cl. CPC A61M 5/2448; A61M 5/19; A61M 5/3159; A61M 2005/31598

(57) ABSTRACT

Mixing systems for producing a mixture to deliver to a treatment site. An illustrative system may comprise a needle hub and a multi-reservoir system. The multi-reservoir system may comprise a first plunger assembly, a second plunger assembly, and a barrel portion. The first plunger assembly, the second plunger assembly, and the barrel portion may be assembled in a telescoping arrangement. Advancing the first plunger assembly may cause a first constituent to be delivered from a first reservoir of the second plunger assembly into the third reservoir to mix with a third constituent to form a precursor and a second constituent to be delivered from a second reservoir of the second plunger assembly into the fourth reservoir and advancing the first and second plunger assembly collectively causes the precursor and the second constituent to be delivered into the needle hub.

### CROSS-REFERENCE TO RELATED APPLICATIONS

This application claims the benefit of U.S. Provisional Patent Application Ser. No. 63/462,139 filed on Apr. 26, 2023, the disclosure of which is incorporated herein by reference.

### FIELD

The present disclosure relates generally to the field of devices for delivering injectable materials and/or compositions to a patient, and associated systems and methods. More particularly, the present disclosure relates to devices for combining constituents of injectable materials and/compositions, and associated systems and methods.

### BACKGROUND

Various forms of cancer and other medical conditions are treated by local application of radiation therapy. However, various risks may accompany radiation therapy. Since the conception of conformal radiotherapy, physicians have paid attention to the radiation dose delivered to the target and surrounding tissues. Investigators have been able to correlate side effects to the amount of tissue receiving a certain radiation dose. And yet, time, distance, and shielding affect the dose that is delivered. The less time an area is exposed to radiation, the less dose is delivered. The greater the distance from the radiation, the less dose is delivered. Filler materials may be injected into a treatment area to provide a shield to tissue surrounding the target of the radiation therapy. For instance, numerous men are diagnosed with prostate cancer each year. Traditionally, treatment options include interstitial implant therapy, surgery, and external beam radiotherapy. While the best treatment is still debatable, side effects of treating prostate cancer have become less toxic with implant therapy and radiotherapy. Various systems provide filler material to treatment sites to decrease the radiation dose to tissue surrounding radiation target sits (e.g., to shield the rectum during radiotherapy for prostate cancer). Such filler materials are often reactive, and therefore are generally combined/mixed immediately prior to or even during delivery to the patient.

Various systems are known for combining/mixing (e.g., in vitro) filler materials injected into radiation treatment areas. However, most such systems include numerous subcomponents, are complex to assemble, and are susceptible to filler mixing errors prior to delivery within a patient at a treatment site. Various challenges posed by such mixing systems may result in errors and mishaps which lead unnecessarily to increased procedure time and increased procedure costs. Solutions to these and other issues presented by combining and delivering injectable materials would be welcome in the art.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with certain embodiments of the present disclosure, devices, systems, and methods for combining and/or delivering injectable materials are disclosed.

In a first example, a system for producing a mixture to deliver to a treatment site may comprise a needle hub and a multi-reservoir system. The multi-reservoir system may comprise a first plunger assembly including a first plunger and a second plunger, a second plunger assembly including a first barrel defining a first reservoir, a second barrel defining a second reservoir, a first flow control member disposed within the first reservoir, and a second flow control member disposed within the second reservoir, the first reservoir configured to receive a portion of the first plunger and a first constituent and the second reservoir configured to receive a portion of the second plunger and a second constituent, wherein the first and second constituents are fluid constituents, and a barrel portion including a housing defining a third reservoir in selective fluid communication with the first reservoir, the third reservoir configured to contain a third constituent, and a fourth reservoir in selective fluid communication with the second reservoir. Actuating the first plunger assembly relative to the second plunger assembly may cause the first constituent to be delivered into the third reservoir to mix with the third constituent and form a precursor and the second constituent to be delivered into the fourth reservoir and wherein actuating the first and second plunger assembly collectively relative to the barrel portion may cause the precursor and the second constituent to be delivered into the needle hub.

Alternatively or additionally to any of the examples above, in another example, the system may further comprise a removable retainer positioned between a distal end of the first plunger assembly and a distal end of the second plunger assembly, the removable retainer may prevent actuation of the first plunger assembly relative to the second plunger assembly.

Alternatively or additionally to any of the examples above, in another example, the system may further comprise a cap removably coupled with a distal end region of the barrel portion.

Alternatively or additionally to any of the examples above, in another example, the first plunger assembly may be configured to be actuated with the cap coupled to the distal end region of the barrel portion.

Alternatively or additionally to any of the examples above, in another example, the first and second plunger assemblies may be configured to be actuated collectively with the distal end region of the barrel portion free from the cap.

Alternatively or additionally to any of the examples above, in another example, the first and second flow control members may be configured to toggle in response to actuating the first plunger assembly relative to the second plunger assembly.

Alternatively or additionally to any of the examples above, in another example, the first flow control member may be positioned between a proximal end and a distal end of the first reservoir and the second flow control member may be positioned between a proximal end and a distal end of the second reservoir.

Alternatively or additionally to any of the examples above, in another example, prior to actuating the first plunger assembly relative to the second plunger assembly, the first constituent may be disposed between a proximal end of the first flow control member and a proximal end of the first reservoir and the second constituent may be disposed between a proximal end of the second flow control member and a proximal end of the second reservoir.

Alternatively or additionally to any of the examples above, in another example, the system may further comprise a needle that is configured to be coupled to the needle hub.

Alternatively or additionally to any of the examples above, in another example, the needle hub may comprise a first lumen in fluid communication with the third reservoir of the barrel portion, a second lumen in fluid communication with the fourth reservoir of the barrel portion, a central lumen configured to be in fluid communication with a needle, and a mixing region connecting the first and second lumens with the central lumen.

Alternatively or additionally to any of the examples above, in another example, the needle hub may be removably coupled to a distal end region of the barrel portion of the multi-reservoir system.

Alternatively or additionally to any of the examples above, in another example, the first plunger assembly, the second plunger assembly, and the barrel portion may be assembled in a telescoping arrangement.

In another example, a method for producing a mixture with a mixing system to deliver to a treatment site may comprise actuating a first plunger assembly within a second plunger assembly to move a first constituent from a first reservoir of the second plunger assembly to third reservoir of a barrel portion and to move a second constituent from a second reservoir of the second plunger assembly to a fourth reservoir of the barrel portion, wherein the first and second constituents are fluid constituents, shaking the first plunger assembly, the second plunger assembly, and the barrel portion to mix the first constituent with a third constituent disposed within the third reservoir of the barrel to form a precursor, coupling a needle hub having a mixing region to the distal end of the barrel portion, actuating the first plunger assembly and the second plunger assembly together to move the precursor and the second constituent into the mixing region of the needle hub to form an injectable mixture.

Alternatively or additionally to any of the examples above, in another example, the method may further comprise removing a retainer from the first plunger assembly prior to actuating the first plunger assembly.

Alternatively or additionally to any of the examples above, in another example, the method may further comprise actuating the first plunger assembly and the second plunger assembly together after removing the cap and prior to coupling the needle hub to the distal end of the barrel portion to purge air from barrel portion.

Alternatively or additionally to any of the examples above, in another example, the method may further comprise removing a cap from a distal end of the barrel portion prior to coupling the needle hub to the distal end of the barrel portion.

In another example, a kit for producing a mixture for delivery to a treatment site may comprise a multi-reservoir system and an injection system. The multi-reservoir system may comprise a first plunger assembly including a first plunger and a second plunger, a second plunger assembly including a first barrel defining a first reservoir, a second barrel defining a second reservoir, a first flow control member disposed within the first reservoir, and a second flow control member disposed within the second reservoir, the first reservoir configured to receive a portion of the first plunger and a first constituent and the second reservoir configured to receive a portion of the second plunger and a second constituent, wherein the first and second constituents are fluid constituents, and a barrel portion including a housing defining a third reservoir in selective fluid communication with the first reservoir, the third reservoir configured to contain a third constituent, and a fourth reservoir in selective fluid communication with the second reservoir. The injection system may comprise a needle hub and a needle coupled to the needle hub.

Alternatively or additionally to any of the examples above, in another example, the kit may further comprise a removable retainer positioned between a distal end of the first plunger assembly and a distal end of the second plunger assembly, the removable retainer configured to prevent actuation of the first plunger assembly relative to the second plunger assembly.

Alternatively or additionally to any of the examples above, in another example, the kit may further comprise a cap removably coupled with a distal end region of the barrel portion.

Alternatively or additionally to any of the examples above, in another example, the kit may further comprise a connector, the connector configured to couple the needle hub to a syringe.

Alternatively or additionally to any of the examples above, in another example, the connector may include a first fluid inlet, a first fluid outlet, and a second fluid outlet.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to facilitate injection into a patient. For purposes of clarity and simplicity, not every element is labeled in every figure, nor is every element of each embodiment shown where illustration is not necessary to allow those of ordinary skill in the art to understand the disclosure. Moreover, reference characters may indicate elements in some figures which are illustrated in other figures, and which, for the sake of brevity, are described only with reference to the other figures.

The detailed description will be better understood in conjunction with the accompanying drawings, wherein like reference characters represent like elements, as follows:

FIG. 1 depicts an exploded perspective view of an illustrative mixing system for mixing an injectable material;

FIG. 2, depicts a cross-sectional view of the multi-reservoir system of the mixing system of FIG. 1, taken at line 2-2;

FIG. 3 depicts a perspective view of the illustrative multi-reservoir system with the retainer removed;

FIG. 4 depicts a cross-sectional view of the multi-reservoir system as the first plunger assembly is axially displaced;

FIG. 5, depicts a cross-sectional view of the illustrative multi-reservoir system with the first plunger assembly fully actuated into the second plunger assembly;

FIG. 6 depicts a perspective view of the illustrative multi-reservoir system being shaken;

FIG. 7 depicts a side view of the illustrative multi-reservoir system with the cap removed;

FIG. 8 depicts a side view of the illustrative multi-reservoir system as air is being purged;

FIG. 9A depicts an exploded perspective view of the illustrative injection system and saline syringe;

FIG. 9B depicts a perspective view of the assembled illustrative injection system and saline syringe;

FIG. 9C depicts a perspective view of the unassembled illustrative injection system and saline syringe;

FIG. 10 depicts a side view of the unassembled injection system and multi-reservoir system;

FIG. 11 depicts a side view of the assembled injection system and multi-reservoir system;

FIG. 12 depicts a cross-sectional view of the assembled injection system and multi-reservoir system taken at line 12-12 of FIG. 11; and

FIG. 13 depicts a cross-sectional view of the assembled injection system multi-reservoir system in a partially dispensed configuration.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location.

Various medical procedures involve delivery (e.g., injection) of injectable material(s) into the body before, during, or after the procedure. Preferably, the injectable material is biocompatible, and optionally biodegradable. The injectable material may serve a variety of purposes, including, without limitation, differentiating tissue (e.g., by creating a “bleb” or other raised or swelled region to distinguish an anatomical region), spacing anatomical structures from one another, otherwise affecting (e.g., shielding, coating, covering, modifying, etc.) an anatomical structure, etc. It will be appreciated that the term “tissue” is a broad term that encompasses a portion of or site within a body: for example, a group of cells, a group of cells and interstitial matter, an organ, a portion of an organ, an anatomical portion of a body, e.g., a rectum, ovary, prostate, nerve, cartilage, bone, brain, or portion thereof, etc. Moreover, reference may be made, herein, to a “target” in referring to an area of the patient's body at which a procedure is to be performed. However, it will be appreciated that such reference is to be broadly understood and is not intended to be limited to tissue or to particular procedures. Finally, reference may be made to target tissue, target location, target site, target tissue site, anatomical site, delivery site, deployment site, injection site, treatment site, etc., including combinations thereof and other grammatical forms thereof, interchangeably and without intent to limit.

Certain specific aspects of the present disclosure relate to placing an injectable material between target tissue to be treated and other tissues. For the sake of convenience, and without intent to limit, reference is made to an injectable material, such as a filler, including, without limitation, a gel composition. The injectable material may be delivered within the patient to displace the tissue relative to a tissue that is to be treated by a therapeutic procedure or otherwise (e.g., not necessarily therapeutic). Certain aspects of the present disclosure include displacing and/or shielding a tissue to protect the tissue against possible side effects of treatment of a target tissue, such as the effects of a treatment involving radiation or cryotherapy. In some aspects, the injectable material may displace anatomical tissue and/or may increase the distance between the target tissue and other tissues. For instance, if the target tissue is to be irradiated, the injectable material may space other tissues from the target tissue so that the other tissues are exposed to less radiation and/or are shielded from the radiation. In some aspects, the injectable material is injected as a filler in a space between tissues. A first tissue may then be treated by radiation, while the injectable material reduces passage of radiation therethrough into a second tissue. The first tissue may be irradiated while the second tissue, spaced by the injectable material, receives less radiation than it would have in the absence of the injectable material. An effective amount of an injectable material may be injected into a space between a first tissue to be treated and a second tissue which can be a critically sensitive organ. For instance, in the context of treatment of prostate cancer, an injectable material may be injected into the Denonvilliers' space (a region between the rectum and prostate) to create additional space between the rectum and prostate and/or to shield the rectum during treatment, thereby reducing rectal radiation dose and associated side effects.

In some aspects of the present disclosure, constituents of an injectable material are combined by a system formed in accordance with various principles of the present disclosure and injected into or near a target site. It will be appreciated that terms such as combine, mix, blend, etc., (including other grammatical forms thereof) may be used interchangeably herein without intent to limit unless otherwise indicated. Reference is accordingly made herein to a combining system generically, without intent to specifically require active combining/mixing.

In accordance with various principles of the present disclosure, the injectable material may be a filler such as a hydrophilic polymer, a gel, a hydrogel, etc. For instance, the injectable material can include polymeric materials which are capable of forming a hydrogel upon crosslinking. Optionally, the polymer forms a hydrogel within the body. A hydrogel is defined as a substance formed when a polymer (natural or synthetic) is crosslinked via covalent, ionic, or hydrogen bonds to create a three-dimensional structure which entraps water molecules to form a gel. Naturally occurring and synthetic hydrogel forming polymers, polymer mixtures and copolymers may be utilized as hydrogel precursors. In some aspects, the hydrogel can be formed by a composition formed by two or more constituents/components (e.g., mixing accelerant fluid, diluent, and polyethylene glycol (PEG) together), and may include one or more polysaccharide compounds or a salt thereof. For example, the composition may include a cellulose compound such as carboxymethyl cellulose (CMC) or salt thereof (e.g., CMC sodium), xanthan gum, alginate or a salt thereof (e.g., calcium alginate, such as Ca-alginate beads), chitosan, and/or hyaluronic acid. In some examples, the composition may comprise a mixture of hyaluronic acid and CMC, and/or may be crosslinked with a suitable crosslinking compound, such as butanediol diglycidyl ether (BDDE). In some aspects, the polysaccharide may be a homopolysaccharide or a heteropolysaccharide.

In some aspects of the present disclosure, two or more constituents of an injectable material are provided separately, and are combined by a device, system, and method in accordance with various principles of the present disclosure to form an injectable material to be injected into or near a target site by devices, systems, and methods in accordance with various principles of the present disclosure. The injectable material may be delivered within the patient to displace the tissue relative to a tissue that is to be treated by a therapeutic procedure or otherwise (e.g., not necessarily therapeutic). A composition to be injected into a patient may be a combination of two or more constituents combined by a device, system, or method formed in accordance with various principles of the present disclosure. In accordance with various principles of the present disclosure, a composition to be injected into a patient may be a combination of two or more constituents combined by a device, system, or method such as formed in accordance with various principles of the present disclosure. For instance, devices, systems, or methods of the present disclosure may be used to combine a first constituent and a second constituent for injection into a patient. The first constituent may be a precursor, e.g., a first constituent to be combined with an additional constituent to form the injectable compound. The second constituent may be an accelerator, an activating agent, a catalyst, an initiator, etc., which, upon combination with the precursor, produces the injectable compound, such as by altering the chemical composition or structure of the first constituent or precursor. The constituents may be combined prior (e.g., immediately prior) to delivery (e.g., injection) or as the constituents are being delivered to the patient so that the injectable material does not have time to form into a structure which may be difficult to inject or otherwise deliver to the patient. As such, the combination of the first constituent or precursor and the second constituent may be such that the injectable compound attains its desired properties and/or reaches its final form in situ.

In some embodiments, the injectable material is formed of a first constituent, a second constituent, and a third constituent. For instance, for various reasons it may be desirable to provide a first, precursor constituent in a solid form (e.g., to be more stable for storage and/or transport). The first constituent is combinable with the third constituent, and the thus-formed combined composition (which may be referenced as the precursor) is then combinable with the second constituent once the medical professional is ready to deliver (e.g., inject) the injectable material to the patient. The second constituent may facilitate a crosslinking interaction between the first and third constituents, for example, by initiating or accelerating the crosslinking interaction of the first and third constituents. Typically, one or more of the constituents of the injectable materials are biocompatible polymers. In some aspects, one of the first, second, or third constituents is a reactive polymer, such as a cross-linkable and/or hydrophilic polymer constituent (e.g., polyethylene glycol (PEG)), and one of the first, second, third constituent is a diluent (e.g., mostly water) in which a solid or semi-solid form of the reactive polymer is dissolved or dispersed, and/or with which the reactive polymer is cross-linked (or at least cross-linkable, such as upon further combination with the second constituent), to form a precursor. Another of the first, second, or third constituents may be an accelerator, an accelerant, an activating agent, a catalyst, an initiator, etc. (such terms being used interchangeably herein without intent to limit), combinable and reactive with the precursor to form the desired injectable material. In one example of an embodiment, a first constituent, in the form of a reactive polymer (specifically, PEG) that has been derivatized with reactive electrophilic groups (specifically, succinimide ester groups), is mixed with a third constituent, in the form of a cross-linking agent (specifically, trilysine, which contains multiple nucleophilic groups, specifically, amino groups), under acidic pH conditions where the succinimide ester groups and the amino groups do not react to any significant degree. When this mixture is combined with a second constituent, in the form of an accelerant (specifically, a basic buffer solution), the pH of the resulting mixture becomes basic, at which point the amino groups of the trilysine react with the succinimide ester groups of the PEG to form covalent bonds, thereby crosslinking the PEG and forming a hydrogel. It will be appreciated that reference to “first”, “second”, or “third” is not intended to connote a particular nature of the material or the order in which the material is combined. As such, “first”, “second”, and “third” may be used to reference any of three constituents forming an injectable material in accordance with various principles of the present disclosure. A non-limiting example of such constituents combinable by devices, systems, or methods in accordance with various principles of the present disclosure includes a reactive constituent, a diluent with which the reactive constituent is to be combined to form a precursor, and an accelerator combinable with the precursor to form an injectable material. The injectable material is a biocompatible material, such as a polymeric material, such as a filler, or such as a hydrogel.

In some examples, the composition may be or include a gel with a desired gel strength and/or viscosity, such as a biocompatible gel suitable for injection (e.g., through a needle), as discussed in further detail below. In one example of an embodiment, the first constituent is a biocompatible polymeric constituent. More particularly, in one example of an embodiment, the first constituent is a hydrophilic polymer, which may be natural or synthetic in origin, and may be anionic, cationic, zwitterionic, or neutrally charged. Non-limiting examples of hydrophilic polymers include natural hydrophilic polymers including proteins such as collagen and polysaccharides such as gellan gum, xanthan gum, gum arabic, guar gum, locust bean gum, alginate, and carrageenans, and synthetic hydrophilic polymers such as polyethylene glycols (PEG), PEG-methacrylates, PEG-methylmethacrylates, polyvinyl alcohols, polyacrylates and polymethacrylates, polyacrylic acids and their salts, polymethacrylic acids and their salts, polymethylmethacrylates, carboxymethylcelluloses, hydroxyethylcelluloses, polyvinylpyrrolidones, polyacrylamides such as N,N-methylene-bis-acrylamides or tris (hydroxymethyl) methacrylamides. The hydrophilic polymer may be modified to provide functional groups that are reactive with functional groups of a suitable cross-linking agent, which may be a covalent or ionic cross-linking agent.

The concentrations of gelling agent(s) in a composition formed in accordance with various principles of the present disclosure maybe at least about 0.01% by weight with respect to the total weight of the composition, and at most about 2.0% by weight with respect to the total weight of the composition, including increments of about 0.01% therebetween. For instance, the concentration of gelling agent(s) may range from about 0.02% to about 1.5%, from about 0.05% to about 1.0%, from about 0.05% to about 0.50%, from 0.05% to about 0.15%, from about 0.10% to about 0.20%, from about 0.15% to about 0.25%, from about 0.20% to about 0.30%, from about 0.25% to about 0.35%, from about 0.30% to about 0.40%, from about 0.35% to about 0.45%, from about 0.40% to about 0.50%, from about 0.1% to about 0.5%, or from about 0.1% to about 0.15% by weight with respect to the total weight of the composition. In at least one example, the total concentration of the gelling agent(s) in the composition may range from about 0.05% to about 0.5% by weight with respect to the total weight of the composition.

In some examples, a composition formed in accordance with various principles of the present disclosure may have a viscosity of at least about 0.001 pascal-second (Pa·s), and at most about 0.100 Pa·s at a shear rate of 130 s⁻¹. For instance, the composition may have a viscosity ranging from about 0.005 Pa·s to about 0.050 Pa·s, from about 0.010 Pa·s to about 0.050 Pa·s, from about 0.010 Pa·s to about 0.030 Pa·s, from about 0.010 Pa·s to about 0.020 Pa·s, from about 0.020 Pa·s to about 0.030 Pa·s, or from about 0.020 Pa·s to about 0.040 Pa·s at a shear rate of 130 s⁻¹. Thus, for example, the composition may be or comprise a gel having a viscosity of about 0.005 Pa·s, about 0.006 Pa·s, 0.008 Pa·s, about 0.010 Pa·s, about 0.011 Pa·s, about 0.012 Pa·s, about 0.013 Pa·s, about 0.014 Pa·s, about 0.015 Pa·s, about 0.016 Pa·s, about 0.017 Pa·s, about 0.018 Pa·s, about 0.019 Pa·s, about 0.020 Pa·s, about 0.022 Pa·s, about 0.024 Pa·s, about 0.026 Pa·s, about 0.028 Pa·s, about 0.030 Pa·s, about 0.032 Pa·s, about 0.034 Pa·s, about 0.036 Pa·s, about 0.038 Pa·s, about 0.040 Pa·s, about 0.042 Pa·s, about 0.044 Pa·s, about 0.046 Pa·s, about 0.048 Pa·s, or about 0.050 Pa·s at a shear rate of 130 s⁻¹. In at least one example, the composition may have a viscosity greater than 0.0050 Pa·s at a shear rate of 130 s⁻¹, e.g., a viscosity ranging from about 0.005 Pa·s to about 0.050 Pa·s, at a shear rate of 130 s⁻¹. In at least one example, the composition may have a viscosity greater than 0.010 Pa·s at a shear rate of 130 s⁻¹, e.g., a viscosity ranging from about 0.010 Pa·s to about 0.030 Pa·s, at a shear rate of 130 s⁻¹.

Alternatively or additionally, a composition formed in accordance with various principles of the present disclosure may have a viscosity of at least about 0.001 Pa·s, and at most about 0.050 Pa·s at a shear rate of 768 s⁻¹. For instance, the composition may have a viscosity ranging from about 0.002 Pa·s to about 0.030 Pa·s, from about 0.003 Pa·s to about 0.020 Pa·s, from about 0.004 Pa·s to about 0.010 Pa·s, from about 0.004 Pa·s to about 0.006 Pa·s, from about 0.005 Pa·s to about 0.007 Pa·s, from about 0.006 Pa·s to about 0.008 Pa·s, from about 0.007 Pa·s to about 0.009 Pa·s, or from about 0.008 Pa·s to about 0.01 Pa·s at a shear rate of 768 s⁻¹. Thus, for example, the composition may be or comprise a gel having a viscosity of about 0.003 Pa·s, about 0.004 Pa·s, about 0.005 Pa·s, about 0.006 Pa·s, about 0.007 Pa·s, about 0.008 Pa·s, about 0.009 Pa·s, or about 0.010 Pa·s at a shear rate of 768 s⁻¹. In at least one example, the composition may have a viscosity less than 0.010 Pa·s at a shear rate of 768 s⁻¹, e.g., a viscosity ranging from about 0.005 Pa·s to about 0.009 Pa·s at a shear rate of 768 s⁻¹. In at least one example, the composition may have a viscosity ranging from about 0.004 Pa·s to about 0.010 Pa·s at a shear rate of 768 s⁻¹. Further, for example, the composition may have a viscosity ranging from about 0.010 Pa·s to about 0.030 Pa·s, e.g., about 0.017 Pa·s at a shear rate of 130 s⁻¹ and a viscosity ranging from about 0.004 Pa·s to about 0.010 Pa·s, e.g., about 0.007 Pa·s, at a shear rate of 768 s⁻¹.

In some embodiments, a multi-reservoir system includes separate reservoirs for constituents to be combined to form the injectable material to be delivered to the patient by an injection system. In some embodiments, a first constituent and a second constituent are separately contained within a first reservoir and a second reservoir, respectively, of a multi-reservoir device. A third constituent may be contained in a separate reservoir defining a third reservoir of the multi-reservoir system. To deliver the injectable material, the constituents of the first and third reservoirs are combined within the third reservoir (e.g., to form a precursor) while the constituent of the second reservoir is moved to a fourth reservoir and then the constituents of the third and fourth reservoirs are injected together into the patient. The multi-reservoir device may or may not mix the contents of the first reservoir with the contents of the second reservoir. For instance, the multi-reservoir device may deliver and inject the constituents of the injectable material to an injection system, with the injection system including a mixer component configured to mix the constituents from the first reservoir of the multi-reservoir device with the contents from the second reservoir of the multi-reservoir device as those contents are injected from the multi-reservoir device and the injection system into the patient. The already-combined first and third constituents are combined with the second constituent to form the desired form, structure, composition, properties, etc., of the injectable material to be delivered and deposited within the patient. The final form, structure, composition, properties, etc., of the injectable material may be attained once the combined constituents are within the patient.

The present disclosure provides devices, systems, and methods for combining constituents to form an injectable composition, and corresponding medical devices, systems, and methods for use thereof and/or delivery to a treatment site of a patient. According to some aspects of the present disclosure, such as described above, a multi-reservoir system may include a plurality of reservoirs for the one or more constituents of the injectable material and for combinations of such constituents. It will be appreciated that terms such as chamber, reservoir, container, vial, lumen, etc., may be used interchangeably herein without intent to limit, to refer to elements which contain, convey, hold, transport, collect, etc., a constituent (fluid, particulate, liquid, solid, gas, etc.) of an injectable material. Suitable chambers may include, for example, vials, syringes (e.g., a syringe barrel compatible with a manual or automatic injection system) and other fluid containers, such as configured for use with a suitable injection system. Examples of materials suitable for the reservoir of devices or systems of the present disclosure include, but are not limited to, cyclic olefin polymer, polypropylene, polycarbonate, polyvinyl chloride, and glass. In some aspects, one of these materials (e.g., cyclic olefin copolymer specifically) can have a coating applied to it (such as SiO₂ coating), which is advantageous so the coating can provide a primary oxygen barrier, behave as a glass-like layer, and/or can be applied using a vapor deposition process.

A combining device or system formed in accordance with various principles of the present disclosure to combine two or more constituents to form an injectable material may include and/or be removably connected to one or more injection systems which are configured to deliver the injectable materials to a patient. According to some aspects of the present disclosure, the filler compositions which may be used with various systems disclosed herein, e.g., the compositions prepared by the various devices, systems, methods disclosed herein, may have sufficient strength, e.g., gel strength, to withstand the forces on the continuity of the three-dimensional configuration (e.g., gel network) of the composition, and thereby minimize the effects of such forces. In the meantime, compositions with sufficient strength to withstand forces thereon may have a viscosity suitable for injection, e.g., a viscosity that does not cause the composition to become stuck in the reservoir(s), delivery lumen, needle, or other structure in which the composition is contained or through which it passes. According to some aspects of the present disclosure, the composition may maintain its three-dimensional structure until the composition is injected into a patient (e.g., through a needle), whereupon the structure may form fragments of the original continuous, three-dimensional network. Those fragments may have a diameter corresponding to the diameter of the lumen through which it passes into the patient (e.g., the lumen of an injection needle), such that the fragments are as large as possible in-vivo to retain as much of the three-dimensional structure of the composition as possible. Injection of these larger-sized particles or fragments is believed to increase the amount of time the gel remains within the tissue.

In some examples, the injection system includes a needle. In some embodiments, the needle may be a hypodermic needle, and may range from a size of 7-gauge (4.57 mm outer diameter (OD), 3.81 mm inner diameter (ID)) to 33-gauge (0.18 mm OD, 0.08 mm ID), e.g., a size of 16-gauge (1.65 mm OD, 1.19 mm ID), 18-gauge, 21-gauge (0.82 mm OD, 0.51 mm ID), 22-gauge (0.72 mm OD, 0.41 mm ID), 23-gauge (0.64 mm OD, 0.33 mm ID), or 24-gauge (0.57 mm OD, 0.31 mm ID). According to some aspects of the present disclosure, the size of the needle may be chosen based on the viscosity and/or constituents of the composition, or vice versa. According to some aspects of the present disclosure, the size of the needle may be 23-gauge or 25-gauge. In some cases, a larger size of 18-gauge, 20-gauge, 21-gauge, or 22-gauge may be used to inject the compositions disclosed herein. Examples of materials which may be used to form the needle include, but are not limited to, metals and metal alloys, such as stainless steel and Nitinol, and polymers. The distal tip of the needle may be sharpened, and may have a beveled shape. The proximal end of the needle may include a suitable fitting/adaptor (e.g., a Luer adapter) for engagement with a syringe or other reservoir. In some examples, the needle may include an elongated tube or catheter between the needle tip and the proximal fitting/adapter.

As noted above, compositions used with systems disclosed herein may have large particulate matter (relative to the injection system lumen) and/or a high viscosity for passage through a lumen sized to inject the material into the patient. The amount of force required to move the composition through a needle aperture (generally described as “peak load” force) may depend on the viscosity of the composition, the dimensions of the needle (inner diameter, outer diameter, and/or length), and/or the material(s) from which the needle is formed. For example, a greater amount of force may be applied to inject the composition through a 33-gauge needle in comparison to a 7-gauge needle. Additional factors that may affect the amount of force applied to inject the composition may include the dimensions of a catheter (inner diameter, outer diameter, and/or length) connecting the mixing system to the needle. Suitable peak loads for injection with one or two hands may range from about 5 pound-force (lb·f) to about 25 lb·f, such as from about 10 lb·f to about 20 lb·f, e.g., about 15 lb·f. The loads measured for a given gel concentration may vary for different needles and flow rates.

According to some aspects of the present disclosure, the size of the needle may be chosen based on the viscosity and/or constituents of the composition, or vice versa. According to some aspects of the present disclosure, the size of the needle may be 23-gauge or 25-gauge. In some cases, a larger size of 18-gauge, 20-gauge, 21-gauge, or 22-gauge may be used to inject the compositions disclosed herein.

According to some aspects of the present disclosure, a combining device or system can be included in a kit for introducing an injectable material into a patient, whereby the injectable material can include any of a variety of suitable compositions. Kits or systems may be configured to store one or more of the constituents of a composition until the medical professional is ready to mix the composition for delivery to a patient. For instance, compositions, such as hydrogels, may be prepared so that the precursor(s) and any related activating agent(s) are stored in the kit with diluents as may be needed. Applicators may be used in combination with the same. Kits formed in accordance with various principles of the present disclosure can be manufactured using medically acceptable conditions and contain constituents that have sterility, purity, and preparation that is pharmaceutically acceptable. Solvents/solutions may be provided in the kit or separately. The kit may include one or more syringes and/or needles for mixing and/or delivery of the injectable material, and/or for additional aspects of the procedure in which the injectable material is to be used. The kit or system may comprise various constituents as set forth herein. For instance, a target site into which an injectable material is to be delivered may be pre-treated using one or more constituents of the kit. One example of a pretreatment includes hydrodissection, such as with saline, to create space for injectable material to be injected at or in the vicinity of the target tissue site. Once saline has been injected to the treatment site, a combining device or system can be connected to a needle (e.g., an 18-gauge spinal needle) to then deliver the injectable material to the treatment site. For instance, in treating prostate cancer, a 5-10 mm layer of filler (e.g., gel composition) may be injected along the posterior wall of the prostate between the prostate and rectum. Once the filler has been injected into the space between the rectum and prostate, ultrasound images can be obtained.

In accordance with various principles of the present disclosure, a combining and/or delivery system is configured to facilitate combining of constituents of an injectable material. In some aspects, the injectable material is a combination of a first constituent, a second constituent, and a third constituent, such as described above. A combining and/or delivery system formed in accordance with various principles of the present disclosure facilitates combining/mixing of the various constituents of an injectable material. Additionally or alternatively, a combining and/or delivery system formed in accordance with various principles of the present disclosure facilitates delivery of the injectable material to an injection system configured to deliver (e.g., inject) and/or deposit the injectable material into a patient (e.g., to a target site within the patient). More particularly, various aspects of the present disclosure simplify assembly, alignment, mixing, dispensing, etc., of an injectable material which is combined or blended or mixed from separate constituents before delivery to a patient.

In some aspects, a combining and/or delivery system formed in accordance with various principles of the present disclosure includes three distinct lumens, chambers, or reservoirs for respective first, second, and third constituents of an injectable material to be delivered and injected into a patient. The present disclosure facilitates combination of the separately-provided first and third constituents before a procedure, as well as combination of the recently-combined first and third constituents with the second constituent for injection into the patient. In particular, an example of an embodiment of a mixing and/or delivery system disclosed herein includes a combining system configured to facilitate the combining of the first, second, and third constituents of an injectable material before delivery to a patient. As described above, it may be desirable to provide the first, second, and third constituents separate from one another for combination only once a procedure is to be performed utilizing the injectable material formed by combining the first, second, and third constituents.

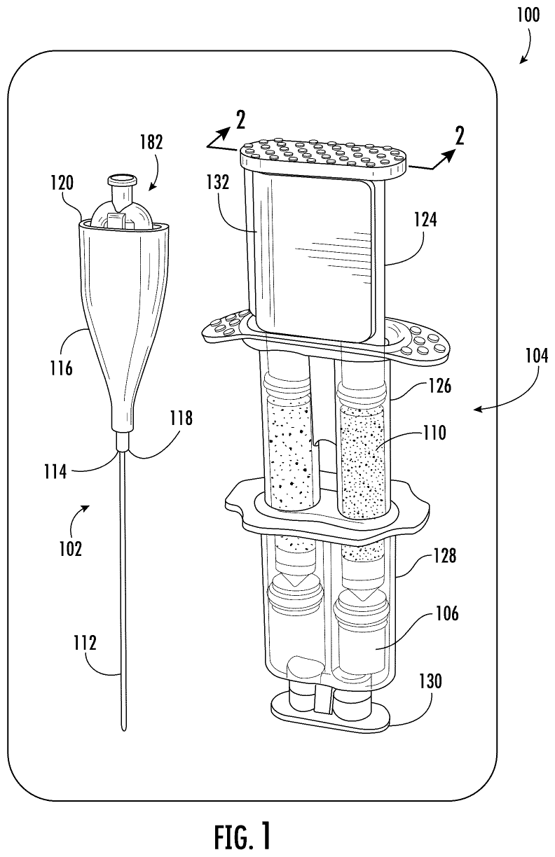

Turning to the drawings, FIG. 1 depicts an exploded perspective view of an illustrative combining and/or delivery system 100 in accordance with certain aspects of the present disclosure for mixing an injectable material. In some embodiments, the combining and/or delivery system 100 may be provided as a kit which may include, but is not limited to, a needle assembly or injection system 102 releasably attachable to a multi-reservoir system 104, a first constituent 106 disposed within a chamber or lumen of the multi-reservoir system 104, a second constituent 108 disposed within another chamber or lumen of the multi-reservoir system 104, and a third constituent 110 disposed within another chamber or lumen of the multi-reservoir system 104. The multi-reservoir system 104 may be used to transport the constituents 106, 108, 110 of an injectable material to a facility and/or location at which the injectable material is to be delivered into a patient. For the sake of convenience, and without intent to limit, reference may be made to delivery by injection into the patient (e.g., the injectable material is delivered to the patient by being injected into the patient), although the present disclosure need not be so limited. As described above, the first, second, and third constituents 106, 108, 110 may be mixed or combined at the time of treatment to form an injectable material.

The injection system 102 may include a needle 112 which can be any needle of this disclosure suitable for hydrodissection as well as delivering the injectable material (e.g., a gel composition) to the treatment site. A proximal end 114 of the needle 112 can be connected to a distal end 118 of a needle hub 116 (e.g., the needle 112 can be overmolded to connect to the needle hub 116). The proximal end 120 of the needle hub 116 may be attached to a distal end of a connector 182. In some embodiments, it is contemplated that the needle 112 may be replaced with a catheter tube or similar structure to reach a target location deeper in the body.

The multi-reservoir system 104 of the overall system 100 may generally include an upper or first plunger assembly 124, a lower or second plunger assembly 126, a barrel portion 128, a cap 130, and a retainer 132. Briefly, a distal portion of the first plunger assembly 124 may be slidably disposed within a proximal portion of the second plunger assembly 126 and a distal portion of the second plunger assembly 126 may be slidably disposed within a proximal portion of the barrel portion 128. The first plunger assembly 124 and the second plunger assembly 126 may be configured to be actuated in succession to first mix two constituents 106, 110 to form a precursor and then to mix the precursor with an accelerator 108 to form an injectable composition. In some cases, at least some of the mixing may occur within the multi-reservoir system 104 and some of the mixing may occur within the injection system 102, as will be described in more detail herein.

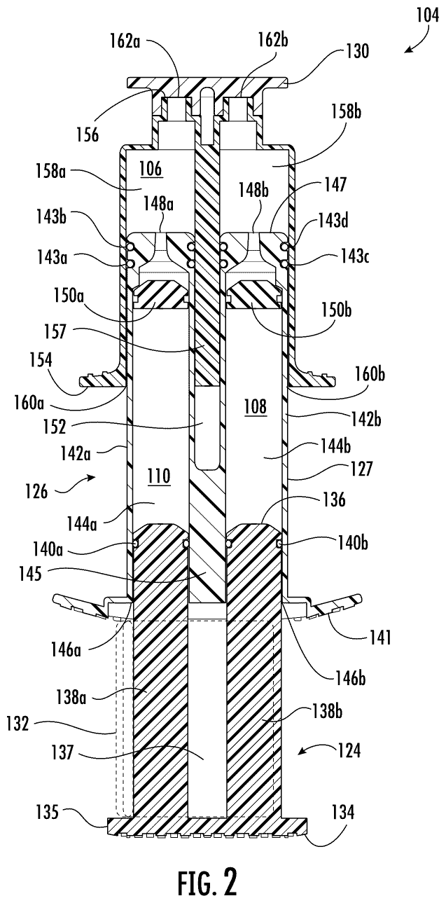

Referring additionally to FIG. 2, which depicts a cross-sectional view of the multi-reservoir system 104 taken at line 2-2 of FIG. 1, the first plunger assembly 124 may extend from a generally planar proximal end 134 to a distal end 136. The proximal end 134 may define a flange or an actuation member 135 for depressing or actuating the first plunger assembly 124. In some cases, a surface of the actuation member 135 may include texturing such as, but not limited to, raised bumps, ridges, etc. to improve the gripability of the proximal end 134. A first plunger 138a and a second plunger 138b may extend distally from actuation member 135 to the distal end 136. The first and second plungers 138a, 138b may be laterally spaced from one another to define a gap 137 therebetween. In some cases, the plungers 138a, 138b may change shape from a proximal end to a distal end thereof. For example, the plungers 138a, 138b may have a first cross-sectional shape adjacent to the proximal end thereof and a second different cross-sectional shape adjacent to the distal end thereof. In some cases, the first cross-sectional shape may be in the shape of a plus sign (e.g., +) while the second cross-sectional shape may be generally circular. These are just examples. Other cross-sectional shapes may be used as desired. It is contemplated that the cross-sectional shape of the distal end of the plungers 138a, 138b may depend at least in part on a cross-sectional shape of the reservoirs 144a, 144b of the second plunger assembly 126. For example, the cross-sectional shape of the distal end of the plungers 138a, 138b may be selected to generally conform to an inner surface of the reservoirs 144a, 144b of the second plunger assembly 126 so as to provide a fluid tight seal therebetween.

Each of the first and second plungers 138a, 138b may include an O-ring 140a, 140b, or other sealing member, such as a gasket, a stopper, etc., disposed about an outer surface thereof adjacent to the distal end 136. The O-ring 140a, 140b may be configured to provide a fluid tight seal between the first plunger assembly 124 and the second plunger assembly 126. In some embodiments, the first plunger assembly 124 may be formed as a single monolithic structure. In other embodiments, the first plunger assembly 124 may be formed as two or more distinct components that are subsequently coupled together. In some cases, the actuation member 135 and the first and second plungers 138a, 138b may be formed as a single monolithic structure and the O-rings 140a, 140b may be provided as separate components. This is just one example. Other configurations may be used, as desired.

The second plunger assembly 126 may include a body portion 127 extending from a proximal end 141 to a distal end 147. The body portion 127 may include a first barrel 142a and a second barrel 142b. The first and second barrels 142a, 142b may extend side-by-side and may be interconnected by a connection portion 145 adjacent the proximal end thereof and laterally spaced adjacent the distal end thereof. The first barrel 142a may define a first chamber, lumen, or reservoir 144a and the second barrel 142b may define a second chamber, lumen, or reservoir 144b. The first and second reservoirs 144a, 144b may each extend through the body portion 127 from a proximal opening 146a, 146b adjacent to the proximal end 141 to a distal opening 148a, 148b adjacent to the distal end 147 of the second plunger assembly 126. In some cases, the first and second reservoirs 144a, 144b may have a same cross-sectional shape and/or dimension along a length thereof. For example, the first and second reservoirs 144a, 144b may have a generally circular cross-sectional shape along a length thereof. However, this is not required. The first and second reservoirs 144a, 144b may take other cross-sectional shapes, as desired. The proximal opening 146a, 146b may be selectively fluidly isolated from the distal opening 148a, 148b via a flow control member 150a, 150b. In the illustrated embodiment, the flow control members 150a, 150b may be a stopper or a gasket. However, this is not required. In some embodiments, the flow control member 150a, 150b may be an out-flow check valve or a one-way valve configured to allow flow in one direction while precluding flow in a second opposing direction.

In some embodiments, the flow control members 150a, 150b may be floating plungers that are not fixedly coupled to any portion of the second plunger assembly 126. As will be described in more detail herein, the flow control members 150a, 150b may be pivotable or toggleable to allow fluid to pass thereby. It is contemplated that the first and second reservoirs 144a, 144b may be fluidly isolated from one another. In some cases, the body portion 127 may define a gap 152 between the distal end regions of the first and second barrels 142a, 142b configured to allow the second plunger assembly 126 to be axially displaced relative to the barrel portion 128, as will be described in more detail herein. Each of the first and second barrels 142a, 142b may include one or more O-rings 143a-d, or other sealing member, such as a gasket, a stopper, etc., disposed about an outer surface thereof adjacent to the distal end 147. The O-rings 143a-d may be configured to provide a fluid tight seal between the second plunger assembly 126 and the barrel portion 128.

In some embodiments, the second plunger assembly 126 may be formed as a single monolithic structure. In other embodiments, the second plunger assembly 126 may be formed as two or more distinct components that are subsequently coupled together. In some cases, the body portion 127, first barrel 142a, and second barrel 142b may be formed as a single monolithic structure and the O-rings 143a-d and/or flow control members 150a, 150b may be provided as separate components. This is just one example. Other configurations may be used, as desired.

The barrel portion 128 may include a body portion 129 extending from a proximal end 154 to a distal end 156. In some examples, the proximal end 154 may include a flange configured to provide a gripping region to allow the user to advance or actuate the first and/or second plunger assemblies 124, 126 relative to the barrel portion 128. In some embodiments, the barrel portion 128 may be formed as a single monolithic structure. In other embodiments, the barrel portion 128 may be formed as two or more distinct components that are subsequently coupled together.

The body portion 129 may define a third chamber, lumen, or reservoir 158a and a fourth chamber, lumen, or reservoir 158b. A wall 157 may separate or fluidly isolate the third reservoir 158a from the fourth reservoir 158b. The third and fourth reservoirs 158a, 158b may each extend through the body portion 129 from a proximal opening 160a, 160b adjacent to the proximal end 154 to a distal opening 162a, 162b adjacent to the distal end 156 of the barrel portion 128. The third and fourth reservoirs 158a, 158b may be sized and shaped to receive at least a distal portion of the second plunger assembly 126 which may be slidably disposed within the third and fourth reservoirs 158a, 158b of the barrel portion 128. In some embodiments, the cross-sectional dimension of the third and fourth reservoirs 158a, 158b may decrease from the proximal end 154 to the distal end 156 of the barrel portion 128. The cross-sectional dimension may decrease in an abrupt step-wise manner to form discrete transitions in the cross-sectional dimension or the cross-sectional dimension may gradually taper.

The reservoirs 144a, 144b, 158a, 158b may be sized to hold a desired volume of respective constituents 106, 108, 110. In some examples, the volume of at least some of the reservoirs 144a, 144b, 158a, 158b may be increased or decreased as the constituents 106, 108, 110 are moved within the multi-reservoir system 104.

A cap 130 may be releasably coupled to the distal end 156 of the barrel portion 128. The cap 130 may be sized and shaped to be disposed over and fluidly seal the distal openings 162a, 162b of the barrel portion 128. While not explicitly shown, the cap 130 may include an elastomeric or deformable sealing material disposed on an interior surface thereof and configured to contact the distal end 156 of the barrel portion 128 to form a fluid tight seal between the cap 130 and the barrel portion 128. The cap 130 may form a snap fit with the distal end 156 of the barrel portion 128. However, other coupling mechanisms may be used, as desired, such as, but not limited to, friction fits, threaded engagements, rotational locks, etc. In some embodiments, the cap 130 may be replaced with a valve or other flow control mechanism configured to selectively fluidly isolate the distal openings 162a, 162b.

The retainer 132 may be removably positioned between a distal side of the actuation member 135 of the first plunger assembly 124 and the proximal end 141 of the second plunger assembly 126. In some cases, the retainer 132 may form a snap fit, or other coupling mechanism with at least one of the first or second plungers 138a, 138b. In other embodiments, the retainer 132 may form a friction fit with the first and/or second plunger assembly 124, 126. While the retainer 132 is positioned between the first plunger assembly 124 and the second plunger assembly 126, distal actuation of the first plunger assembly 124 may be precluded.

Generally, the first plunger assembly 124, the second plunger assembly 126, and the barrel portion 128 may be assembled in a telescoping arrangement. For example, a portion of first plunger assembly 124 may be disposed within a portion of the second plunger assembly 126 and a portion of the second plunger assembly 126 may be disposed within a portion of the barrel portion 128. More particularly, the first plunger assembly 124 may be assembled with the second plunger assembly 126 such that the first plunger 138a of the first plunger assembly 124 is slidably disposed within the first reservoir 144a of the second plunger assembly 126 and the second plunger 138b of the first plunger assembly 124 is slidably disposed within the second reservoir 144b of the second plunger assembly 126. Further, the second plunger assembly 126 may be assembled with the barrel portion 128 such that the first barrel 142a of the second plunger assembly 126 is slidably disposed with the third reservoir 158a of the barrel portion 128 and the second barrel 142b of the second plunger assembly 126 is slidably disposed within the fourth reservoir 158b of the barrel portion 128. The first plunger assembly 124 is movable, such as axially and/or longitudinally slidable, with respect to the second plunger assembly 126 to move (e.g., eject) materials out of and/or to move (e.g., aspirate) materials into first and/or second reservoirs 144a, 144b within the second plunger assembly 126. Similarly, the first plunger assembly 124 and the second plunger assembly 126 may be collectively movable, such as axially and/or longitudinally slidable, with respect to the barrel portion 128 to move (e.g., eject) materials out of and/or to move (e.g., aspirate) materials into third and/or fourth reservoirs 158a, 158b within the barrel portion 128.

The second plunger assembly 126 and the barrel portion 128 may be pre-loaded with the constituents required to form the injectable material. For example, a first constituent, such as, but not limited to, a first cross-linkable constituent 106 may be disposed within the third reservoir 158a of the barrel portion 128. In some examples, the first cross-linkable constituent 106 may be provided as a powder. A second constituent 108, such as, but not limited to, an accelerator, may be disposed within the second reservoir 144b of the second plunger assembly 126. In some examples, the second constituent 108 may be provided as a liquid. A third constituent 110, such as, but not limited to, a second cross-linkable constituent, may be disposed within the first reservoir 144a of the second plunger assembly 126. In some examples, the third constituent 110 may be provided as a liquid. The O-rings 143a, 143b and the flow control member 150a may fluidly isolate the third constituent 110 from the first constituent 106 until mixing is desired. Further, the second constituent 108 may be fluidly isolated from each of the first and third constituents 106, 110 until mixing is desired.

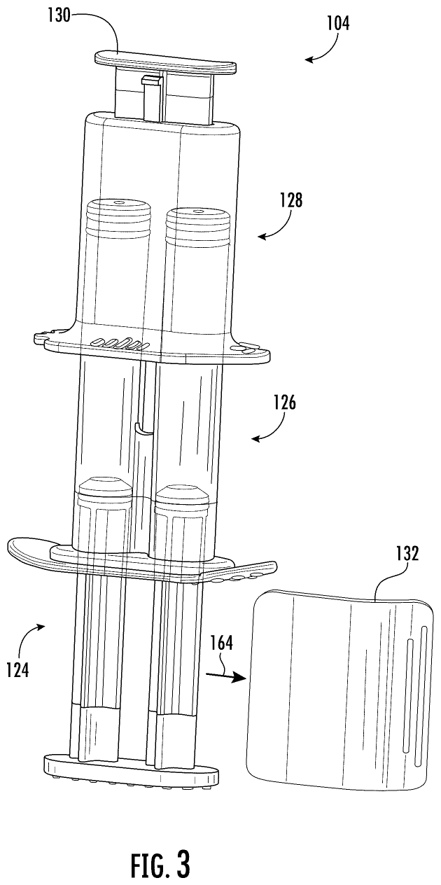

A method for dispensing or injecting the injectable material, along with additional features of the combining and/or delivery system 100 will now be described with respect to FIGS. 3-13. While certain steps are shown as a sequence between each figure, in other embodiments fewer steps are contemplated and the order by which steps are performed can be different than what is illustrated. To begin, the retainer 132 is removed from the first plunger assembly 124, as shown in FIG. 3, which depicts a perspective view of the illustrative multi-reservoir system 104 with the retainer 132 removed. The retainer 132 may be laterally displaced, as shown at arrow 162, to uncouple the retainer 132 from the first plunger assembly 124. However, other movements or actions may be used to uncouple the retainer 132 from the first plunger assembly 124.

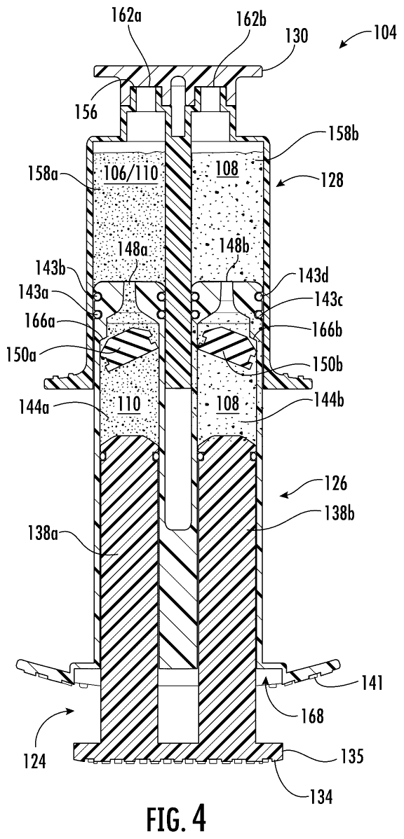

Once the retainer 132 has been removed, the first plunger assembly 124 may be actuated. For example, the first plunger assembly 124 may be pushed or depressed axially towards the distal end 156 of the barrel portion 128. FIG. 4 depicts a cross-sectional view of the multi-reservoir system 104 as the first plunger assembly 124 is being axially displaced. As the first plunger assembly 124 moves distally, the first plunger 138a exerts a force on the third constituent 110, which is stored in the first reservoir 144a proximal to the flow control member 150a, which in turn exerts a force on and actuates or distally advances the flow control member 150a. The first barrel 142a may include a radially inwardly extending ledge or rib 166a along a region thereof. The ledge 166a may extend about less than an entirety of the circumference of the first reservoir 144a such that as the flow control member 150a is actuated or distally advanced, a distal surface of the flow control member 150a contacts the ledge 166a and further axial movement of at least a portion of the flow control member 150a is precluded while another portion of the flow control member 150a may continue to be axially displaced in the absence of a mechanical stop. This may cause the flow control member 150a to toggle or pivot, as shown in FIG. 4. This may cause a previous seal between the flow control member 150a and the reservoir wall to break and create a flow path between the proximal end region of the first reservoir 144a and the distal region of the first reservoir 144a to allow the third constituent 110 to distally advance or flow and pass the flow control member 150a as the first plunger assembly 124 is actuated or distally advanced. The third constituent 110 may exit the distal opening 148a of the first reservoir 144a and enter the third reservoir 158a of the barrel portion 128 where it mixes with the first constituent 106. The ledge 166a and/or flow control member 150a can come in any number of shapes and sizes so as to induce a moment and related toggle action between the ledge 166a and the flow control member 150a. As shown, a tip of flow control member 150a can be pointed or tapered so that contacting the ledge 166a causes a moment and toggling of the flow control member 150a. However, the ledge 166a could be tapered or otherwise include a contoured lower surface configured to induce any shaped tip of flow control member 150a to toggle, as needed or required.

Similarly, as the first plunger assembly 124 moves distally, the second plunger 138b exerts a force on the second constituent 108, which is stored in the second reservoir 144b proximal to the flow control member 150b, which in turn exerts a force on and actuates or distally advances the flow control member 150b. The second barrel 142b may include a radially inwardly extending ledge or rib 166b along a region thereof. The ledge 166b may extend about less than an entirety of the circumference of the second reservoir 144b such that as the flow control member 150b is actuated distally advanced, a distal surface of the flow control member 150b contacts the ledge 166b and further axial movement of at least a portion of the flow control member 150b is precluded while another portion of the flow control member 150b may continue to be axially displaced in the absence of a mechanical stop. This may cause the flow control member 150b to toggle or pivot, as shown in FIG. 4. This may cause a previous seal between the flow control member 150b and the reservoir wall to break and create a flow path between the proximal end region of the second reservoir 144b and the distal end region of the second reservoir 144b to allow the second constituent 108 to distally advance or flow and pass the flow control member 150b as the first plunger assembly 124 is actuated or distally advanced. The second constituent 108 may exit the distal opening 148b of the second reservoir 144b and enter the fourth reservoir 158b of the barrel portion 128. The ledge 166b and/or flow control member 150b can come in any number of shapes and sizes so as to induce a moment and related toggle action between the ledge 166b and the flow control member 150b. As shown, a tip of flow control member 150b can be pointed or tapered so that contacting the ledge 166b causes a moment and toggling of the flow control member 150b. However, the ledge 166b could be tapered or otherwise include a contoured lower surface configured to induce any shaped tip of flow control member 150b to toggle, as needed or required.

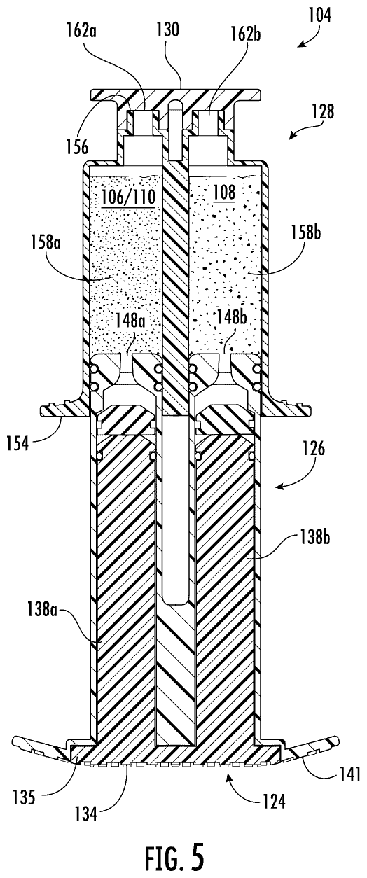

The first plunger assembly 124 may be distally displaced until the actuation member 135 contacts the proximal end 141 of the second plunger assembly 126, as shown in FIG. 5, which depicts a cross-sectional view of the illustrative multi-reservoir system 104 with the first plunger assembly 124 fully actuated into the second plunger assembly 126. As the fluid exits the first and second reservoirs 144a, 144b, the second plunger assembly 126 is proximally displaced within the barrel portion 128 by the fluid entering the third and fourth reservoirs 158a, 158b thereof thus increasing the volume of the third and fourth reservoirs 158a, 158b. The cap 130 may inhibit or prevent fluid from exiting the distal openings 162a, 162b of the third and fourth reservoirs 158a, 158b as fluid is transferred from the second plunger assembly 126 to the barrel portion 128. The first and third constituents 106, 110 may now be in the third reservoir 158a of the barrel portion 128 and the second constituent 108 may now be in the fourth reservoir 158b of the barrel portion 128.

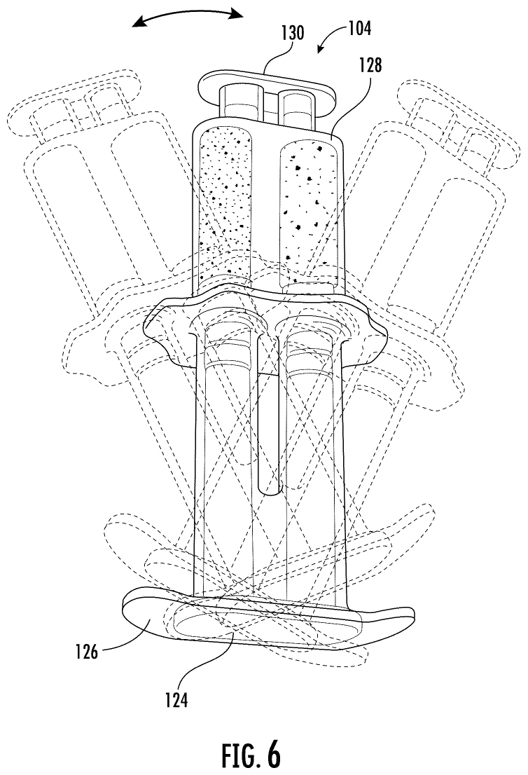

In some cases, the proximal end 141 of the second plunger assembly 126 may include a recess 168 sized and shaped to receive the actuation member 135, although this is not required. When the actuation member 135 is disposed within the recess of the second plunger assembly 126, the proximal end 134 of the first plunger assembly 124 and the proximal end 141 of the second plunger assembly 126 may be generally aligned or flush with one another. Once the first constituent 106 has been injected from the first reservoir 144a into the third reservoir 158a, the multi-reservoir system 104 may be shaken, as shown in FIG. 6, which illustrates a perspective view of the illustrative multi-reservoir system 104 being shaken, to mix the first constituent 106 and the third constituent 110 to form the precursor 106/110. As used herein, the term “fluid” as it relates to constituents 106, 108, 110 of the system 100 is defined broadly and can include liquids, gels, oils, and particulate matter such as granules, pellets, or powders, or any combination of liquids, gels, oils, and/or particulate matter (e.g., granules, pellets, or powders). In some examples, the third constituent 110 can be a diluent fluid solution and the first constituent 106 can include a cross-linkable polymer, such as PEG having a plurality of succinimidyl termini or any other agent mixable with diluent 110 to form precursor. The diluent can include a low molecular weight compound that contains multiple nucleophilic groups, such as trilysine which contains multiple amino groups, dissolved in a low pH (4.0) aqueous solution, though other diluent fluid solutions are contemplated within the scope of this disclosure. Once mixed together, the precursor solution 106/110 can be formed in the third reservoir 158a.

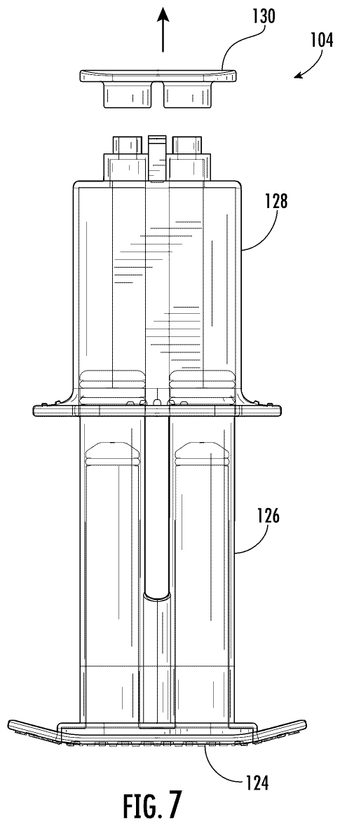

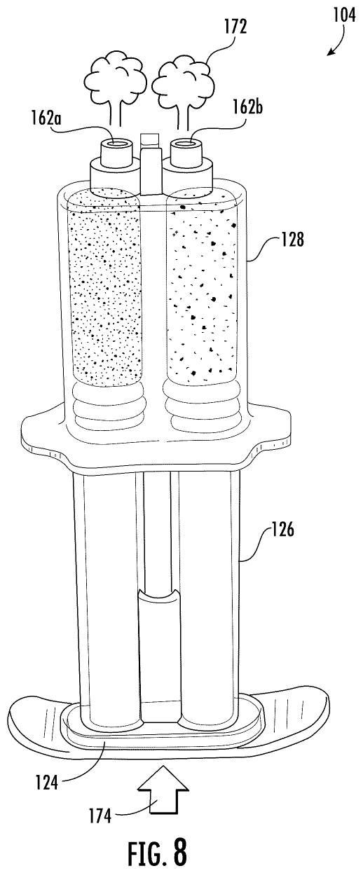

Next, the cap 130 may be removed as shown in FIG. 7 which depicts a side view of the illustrative multi-reservoir system 104 with the cap 130 removed. In some examples, the cap 130 may be axially displaced, as shown at arrow 170. However, this is not required. In some examples, the cap 130 may be removed with other movements and/or forces. After the cap 130 has been removed, the first and second plunger assemblies 124, 126 may be actuated or distally advanced, as shown at arrow 174, to purge air 172 from the multi-reservoir system 104, as shown in FIG. 8, which depicts a side view of the illustrative multi-reservoir system 104, as air is being purged. The multi-reservoir system 104 may be held in an upright manner (e.g., with the distal end 156 of the barrel portion 128 pointed upwards (or away from the ground) to prevent an excess of fluid from dripping and/or leaking from the third and fourth reservoirs 158a, 158b of the barrel portion 128. In some cases, some fluid may be dispensed from the third and fourth reservoirs 158a, 158b during air purging to ensure no air remains in the third and/or fourth reservoirs 158a, 158b.

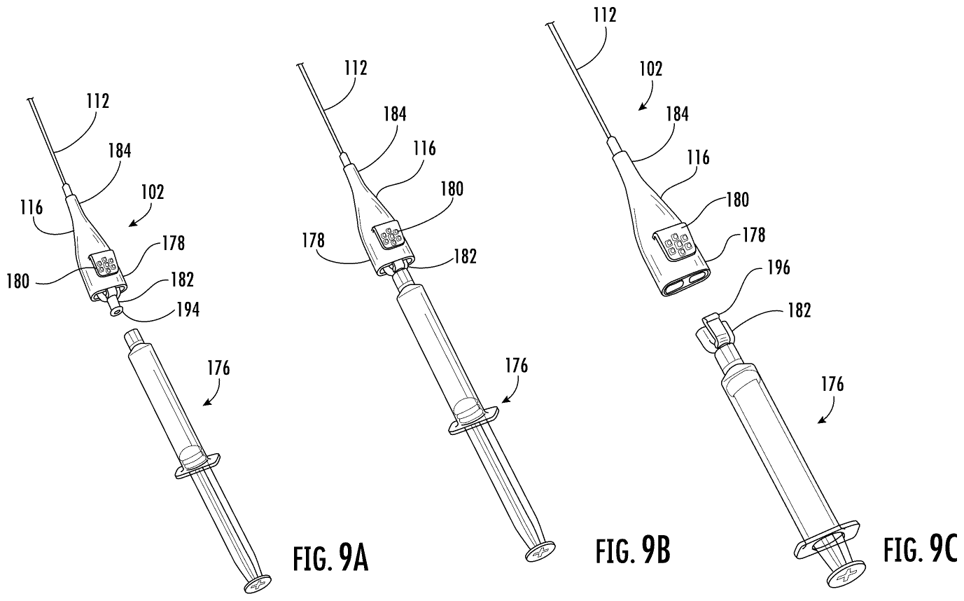

The multi-reservoir system 104 may be maintained in an upright orientation as hydrodissection is performed. Hydrodissection may be optionally performed before injecting the injectable material into the body. FIGS. 9A-9C depict an illustrative method for assembling and disassembling a syringe 176, such as, but not limited to, a saline syringe, for use in hydrodissection, with the injection system 102. Generally, the injection system 102 may be connected to the saline syringe 176. The needle 112 may then be positioned at the treatment site and saline injected to perform hydrodissection. In some examples, the saline syringe 176 may be provided separately from the combining and/or delivery system 100, although this is not required. Once the hydrodissection is complete, the saline syringe 176 may be uncoupled from the injection system 102 with the needle 112 remaining in position and primed at the treatment site (e.g., the distal end of the needle 112 remains in the body). However, this is not required. In some cases, the needle 112 may be removed from the body after hydrodissection.

The needle hub 116 may include a lower housing 178 configured to be gripped and squeezed by a user. One or more externally positioned buttons 180 can be positioned on an outer surface of lower housing 178. In some cases, two buttons 180 may be positioned on opposing sides of the lower housing 178. The button(s) 180 can be configured so that an actuating squeeze or other movement by a user causes latches of an adaptor or connector 182 and/or the barrel portion 128 of the multi-reservoir system 104 to release from mating apertures in the needle hub 116. However, other coupling mechanisms between the needle hub 116 and the connector 182 or barrel portion 128 are contemplated as needed or required. For example, and without limitation, snap fit connectors, magnetic connectors, female-male connectors, hook and loop fasteners, and the like are contemplated.

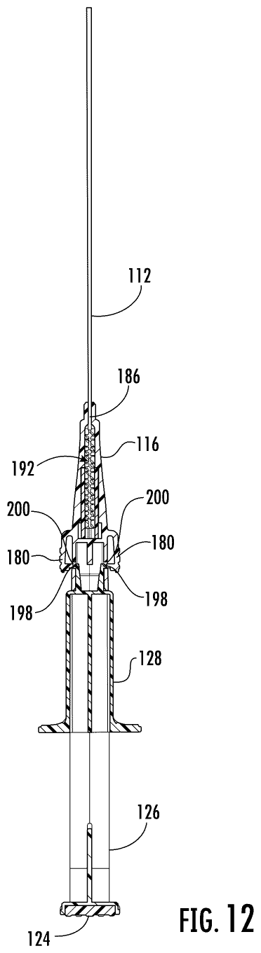

The needle hub 116 may also include a transitional portion 184 through which the needle 112 can be inserted. The transitional portion 184 can include a diameter (or cross-sectional dimension) smaller than the lower housing 178. In some examples, the transitional portion 184 can be tapered and/or include a textured outer surface. A central tubular lumen 186 connected to a first lumen 188 and a second lumen 190 (see, for example, FIG. 12) can pass through the needle hub 116 and be in fluid communication with the needle 112, when the needle 112 is connected to needle hub 116. The first and second lumens 188, 190 may be configured to be in fluid communication with one or more lumens of the connector 182 when connected thereto and to be in fluid communication with the distal openings 162a, 162b of the barrel portion 128 of the multi-reservoir system 104 when connected thereto. The connector 182 may be an adaptor, manifold, etc. configured to fluidly couple the single outlet of the saline syringe 176 to the first and second lumens 188, 190 of the needle hub 116. For example, the connector 182 may have a single fluid inlet for coupling to the outlet of the saline syringe 176 and two or more fluid outlets for coupling to first and second lumens 188, 190 of the needle hub 116. The connector 182 may include more than one fluid inlet or fewer than two or more than two fluid outlets as needed or desired. The first and second lumens 188, 190 of the needle hub 116 may intersect or merge at a mixing region 192. The mixing region 192 may include a static mixer configured to mix the fluids from the first and second lumens 188, 190.

To perform the hydrodissection, the injection system 102 and a saline syringe 176 may be required, as shown in FIG. 9A which depicts an exploded perspective view of the illustrative injection system 102 and saline syringe 176. While the syringe 176 is described as a saline syringe, the syringe 176 may include other fluids, as desired. To connect the injection system 102 with the saline syringe 176, a proximal end 194 of the connector 182, having a single fluid opening, may be aligned with the outlet of the saline syringe 176. The connector 182 may then be connected to the outlet of the saline syringe 176, as shown in FIG. 9B, which depicts a perspective view of the assembled illustrative injection system 102 and saline syringe 176. In some cases, the proximal end 194 of the connector 182 and the outlet of the saline syringe 176 may have mating Luer fittings. However, other connection mechanisms may be used as desired, such as, but not limited to, friction fits, snap fits, threaded engagements, etc. Hydrodissection may then be performed. Once the hydrodissection is complete, the saline syringe 176 may be uncoupled from the injection system 102 while leaving the needle 112 at the treatment site. It is further contemplated that the connector 182 may also be uncoupled from the injection system 102. In some cases, the connector 182 and the saline syringe 176 may be uncoupled from the injection system 102 substantially simultaneously. For example, actuation of the button 180 may cause one or more latches 196 of the connector 182 to release from a mating recess of the needle hub 116. In some cases, a latch 196 may be provided on each of the opposing sides of the connector 182, although this is not required. Proximal retraction of the saline syringe 176 while the button 180 of the needle hub 116 is actuated may release both the connector 182 and the saline syringe 176 from the needle hub 116, as shown in FIG. 9C, which depicts a perspective view of the unassembled illustrative injection system 102 and saline syringe 176. The connector 182 may remain coupled to the saline syringe 176 or may be subsequently uncoupled from the saline syringe 176, if so desired. While the connector 182 and the saline syringe 176 have been described as being uncoupled substantially simultaneously, in some cases, the saline syringe 176 may first be uncoupled from the connector 182 and then the connector 182 uncoupled from the needle hub 116.

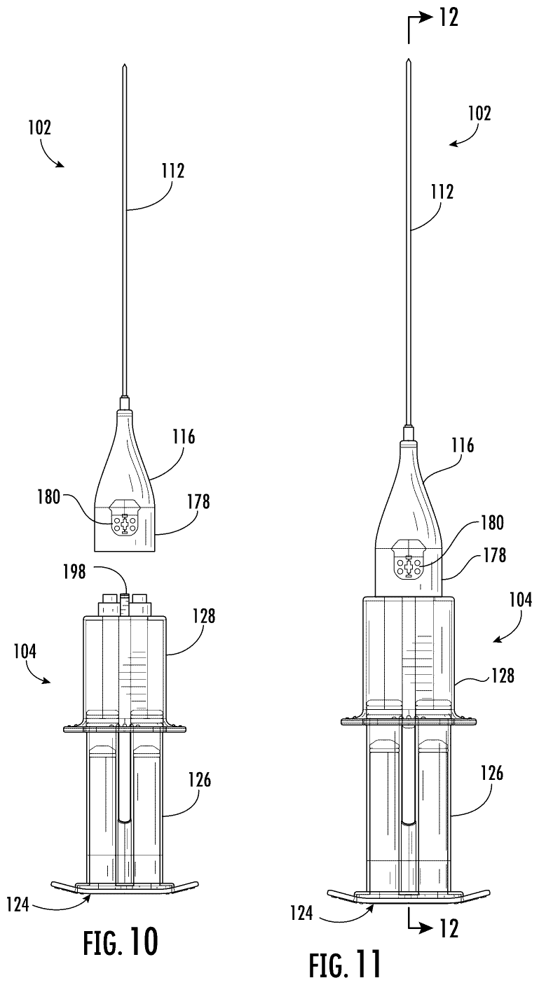

Next, the multi-reservoir system 104 may be connected to the needle hub 116. To do so, the first and second lumens 188, 190 may be aligned with the distal openings 162a, 162b of the barrel portion 128, as shown in FIG. 10 which depicts a side view of the unassembled injection system 102 and multi-reservoir system 104. When assembled, the lower housing 178 of the needle hub 116 may be slid over an outer surface of a distal end region of the barrel portion 128, as shown in FIG. 11 which depicts a side view of the assembled injection system 102 and multi-reservoir system 104. For example, the needle hub 116 may be disposed over the region having the reduced cross-sectional dimension. Referring additionally to FIG. 12 which depicts a cross-sectional view of the assembled injection system 102 and multi-reservoir system 104 taken at line 12-12 of FIG. 11, when the needle hub 116 is assembled with the distal end region of the barrel portion 128, one or more tabs 198 of the barrel portion 128 may be received within one or more mating apertures 200 of the needle hub 116. The one or more apertures 200 may be disposed below the one or more buttons 180 such that actuating (e.g., depressing) the one or more buttons 180 is configured to disengage the one or more tabs 198 of the barrel portion 128 from the one or more mating apertures 200 to allow the injection system 102 to be uncoupled from the multi-reservoir system 104.

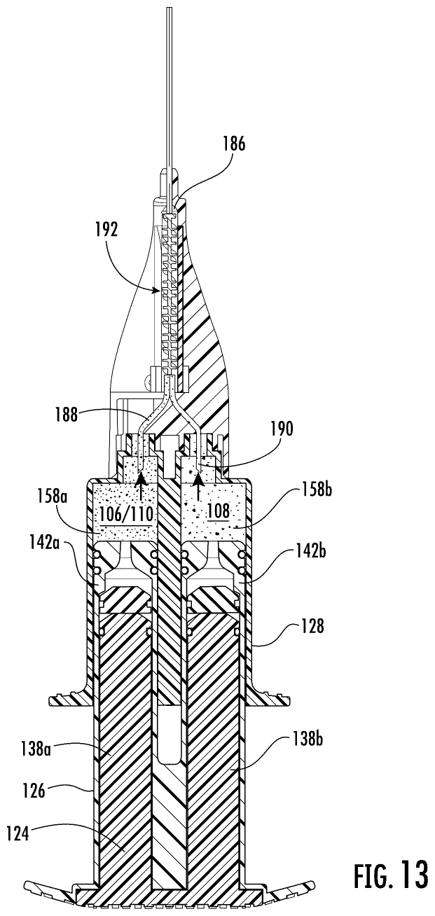

Once the multi-reservoir system 104 has been assembled with the injection system 102, the first and second plunger assemblies 124, 126 can be actuated or distally advanced together to cause the precursor (e.g., resulting from the mixture of the first constituent 106 and the third constituent 110) disposed in the third reservoir 158a of the barrel portion 128 and the second constituent 108 (e.g., the accelerator, such as, but not limited to a basic buffer solution) disposed in the fourth reservoir 158b of the barrel portion 128 to be dispensed from the distal openings 162a, 162b. Referring additionally to FIG. 13 which depicts a cross-sectional view of the assembled injection system 102 and multi-reservoir system 104 in a partially dispensed configuration, the first and second lumens 188, 190 of the needle hub 116 may be in fluid communication with the third and fourth reservoirs 158a, 158b and the distal openings 162a, 162b. In some examples, the pressure of the fluids in the third and fourth reservoirs 158a, 158b may push against the flow control members 150a, 150b to move them into their original (untoggled) configuration. However, this is not required. As the first and second plunger assemblies 124, 126 are actuated or distally advanced or depressed, fluid may exit the third and fourth reservoirs 158a, 158b of the barrel portion 128 and enter the first and second lumens 188, 190 of the needle hub 116. The first and second lumens 188, 190 of the needle hub 116 may be connected with a mixing region 192. The mixing region 192 may be configured to mix or combine the precursor 106/110 and the second constituent 108 prior to the constituents exiting the needle 112. For example, the mixing region 192 may mix or combine the precursor and the second constituent 108 to form the injectable composition before the resulting mixture enters the central lumen 186 of the needle hub 116 which in turn is in fluid communication with a lumen of the needle 112. The injectable composition may continue egressing through the needle 112 and ultimately to the treatment site. The injectable composition formed from the combination of the precursor 106/110 and the second constituent 108 may attain its final desired properties and/or reach its final form in situ or at the target site. In the fully dispensed configuration, the connection portion 145 of the second plunger assembly 126 may contact the wall 157 of the barrel portion 128, although this is not required.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

All apparatuses and methods discussed herein are examples of apparatuses and/or methods implemented in accordance with one or more principles of this disclosure. These examples are not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

In the foregoing description and the following claims, the following will be appreciated. The phrases “at least one”, “one or more”, and “and/or”, as used herein, are open-ended expressions that are both conjunctive and disjunctive in operation. The term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The foregoing discussion has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. One skilled in the art will appreciate that the disclosure may be used with many modifications of structure, arrangement, proportions, materials, components, and otherwise, used in the practice of the disclosure, which are particularly adapted to specific environments and operative requirements without departing from the principles of the present disclosure. For example, elements shown as integrally formed may be constructed of multiple parts or elements shown as multiple parts may be integrally formed, the operation of elements may be reversed or otherwise varied, the size or dimensions of the elements may be varied, and features and components of various embodiments may be selectively combined. The presently disclosed embodiments are therefore to be considered in all respects as illustrative and not restrictive, the scope of the claimed invention being indicated by the appended claims, and not limited to the foregoing description.

The following claims are hereby incorporated into this Detailed Description by this reference, with each claim standing on its own as a separate embodiment of the present disclosure. In the claims, the term “comprises/comprising” does not exclude the presence of other elements or steps. Furthermore, although individually listed, a plurality of means, elements or method steps may be implemented by, e.g., a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second”, etc., do not preclude a plurality. Reference signs in the claims are provided merely as a clarifying example and shall not be construed as limiting the scope of the claims in any way.

What is claimed is:

1. A system for producing a mixture to deliver to a treatment site, comprising:
a needle hub; and
a multi-reservoir system, the multi-reservoir system comprising;
a first plunger assembly including a first plunger and a second plunger;
a second plunger assembly including a first barrel defining a first reservoir, a second barrel defining a second reservoir, a first flow control member disposed within the first reservoir, and a second flow control member disposed within the second reservoir, the first reservoir configured to receive a portion of the first plunger and a first constituent and the second reservoir configured to receive a portion of the second plunger and a second constituent, wherein the first and second constituents are fluid constituents; and
a barrel portion including a housing defining a third reservoir in selective fluid communication with the first reservoir, the third reservoir configured to contain a third constituent, and a fourth reservoir in selective fluid communication with the second reservoir;
wherein actuating the first plunger assembly relative to the second plunger assembly causes the first constituent to be delivered into the third reservoir to mix with the third constituent and form a precursor and the second constituent to be delivered into the fourth reservoir and wherein actuating the first and second plunger assembly collectively relative to the barrel portion causes the precursor and the second constituent to be delivered into the needle hub.

2. The system of claim 1, further comprising a removable retainer positioned between a distal end of the first plunger assembly and a distal end of the second plunger assembly, the removable retainer preventing actuation of the first plunger assembly relative to the second plunger assembly.

3. The system of claim 1, further comprising a cap removably coupled with a distal end region of the barrel portion.

4. The system of claim 3, wherein the first plunger assembly is configured to be actuated with the cap coupled to the distal end region of the barrel portion.

5. The system of claim 3, wherein the first and second plunger assemblies are configured to be actuated collectively with the distal end region of the barrel portion free from the cap.

6. The system of claim 1, wherein the first and second flow control members are configured to toggle in response to actuating the first plunger assembly relative to the second plunger assembly.

7. The system of claim 1, wherein the first flow control member is positioned between a proximal end and a distal end of the first reservoir and the second flow control member is positioned between a proximal end and a distal end of the second reservoir.

8. The system of claim 1, wherein prior to actuating the first plunger assembly relative to the second plunger assembly, the first constituent is disposed between a proximal end of the first flow control member and a proximal end of the first reservoir and the second constituent is disposed between a proximal end of the second flow control member and a proximal end of the second reservoir.

9. The system of claim 1, further comprising a needle that is configured to be coupled to the needle hub.

10. The system of claim 1, wherein the needle hub comprises a first lumen in fluid communication with the third reservoir of the barrel portion, a second lumen in fluid communication with the fourth reservoir of the barrel portion, a central lumen configured to be in fluid communication with a needle, and a mixing region connecting the first and second lumens with the central lumen.

11. The system of claim 1, wherein the needle hub is removably coupled to a distal end region of the barrel portion of the multi-reservoir system.

12. The system of claim 1, wherein the first plunger assembly, the second plunger assembly, and the barrel portion are assembled in a telescoping arrangement.

13. A kit for producing a mixture for delivery to a treatment site, the kit comprising:
a multi-reservoir system, the multi-reservoir system comprising;
a first plunger assembly including a first plunger and a second plunger;
a second plunger assembly including a first barrel defining a first reservoir, a second barrel defining a second reservoir, a first flow control member disposed within the first reservoir, and a second flow control member disposed within the second reservoir, the first reservoir configured to receive a portion of the first plunger and a first constituent and the second reservoir configured to receive a portion of the second plunger and a second constituent, wherein the first and second constituents are fluid constituents; and
a barrel portion including a housing defining a third reservoir in selective fluid communication with the first reservoir, the third reservoir configured to contain a third constituent, and a fourth reservoir in selective fluid communication with the second reservoir; and
an injection system, the injection system comprising:
a needle hub; and
a needle coupled to the needle hub.

14. The kit of claim 13, further comprising a removable retainer positioned between a distal end of the first plunger assembly and a distal end of the second plunger assembly, the removable retainer configured to prevent actuation of the first plunger assembly relative to the second plunger assembly.

15. The kit of claim 13, further comprising a cap removably coupled with a distal end region of the barrel portion.

16. The kit of claim 13, further comprising a connector, the connector configured to couple the needle hub to a syringe.

17. The kit of claim 16, wherein the connector includes a first fluid inlet, a first fluid outlet, and a second fluid outlet.

18. A method for producing a mixture with a mixing system to deliver to a treatment site, the method comprising:
actuating a first plunger assembly within a second plunger assembly to move a first constituent from a first reservoir of the second plunger assembly to third reservoir of a barrel portion and to move a second constituent from a second reservoir of the second plunger assembly to a fourth reservoir of the barrel portion, wherein the first and second constituents are fluid constituents;
shaking the first plunger assembly, the second plunger assembly, and the barrel portion to mix the first constituent with a third constituent disposed within the third reservoir of the barrel portion to form a precursor;
coupling a needle hub having a mixing region to the distal end of the barrel portion; and
actuating the first plunger assembly and the second plunger assembly together to move the precursor and the second constituent into the mixing region of the needle hub to form an injectable mixture.

19. The method of claim 18, further comprising removing a retainer from the first plunger assembly prior to actuating the first plunger assembly.

20. The method of claim 18, further comprising actuating the first plunger assembly and the second plunger assembly together after removing the cap and prior to coupling the needle hub to the distal end of the barrel portion to purge air from barrel portion.
